(19) United States
(12) Patent Application Publication
(10) Pub. No.: US 2009/0209969 A1
(43) Pub. Date: Aug. 20, 2009

(54) EXPANDABLE ACCESS SHEATH

(75) Inventor: Justin Wolfe, Lawrenceville, GA (US)

Correspondence Address: Rutan & Tucker, LLP., 611 ANTON BLVD, SUITE 1400, COSTA MESA, CA, 92626, US

(73) Assignee: C.R. BARD INC, MURRAY HILL, NJ (US)

(21) Appl. No.: 11/817,211

(22) PCT Filed: Mar. 2, 2006
(86) PCT No.: PCT/US2006/007761

§ 371 Date: Jan. 2, 2009

Related U.S. Application Data
(60) Provisional application No. 60/657,904, filed on Mar. 2, 2005.

(51) Int. Cl. A61F 11/00; A61M 29/00

(52) U.S. Cl. 606/108; 604/96.01

(57) ABSTRACT

An expandable access sheath. In one embodiment, the sheath includes a lumen defining an inner passage and an outer surface, and a balloon that is provided on the lumen outer surface, wherein when the balloon is inflated the balloon expands the lumen to increase the size of the inner passage.

### BACKGROUND

Ureteral access sheaths are used to form a channel along a patient's urinary tract that provides access to a location along the tract, such as the ureter. With an established channel to the ureter, a surgeon can insert and withdraw a ureteroscope or other instrument more rapidly and with less trauma to the patient's urinary system.

Typical ureteral access sheaths include two subassemblies: a dilator and a sheath. The dilator is placed within the sheath, and the dilator and sheath are advanced together through the urethra, through the bladder, and into the ureter. The dilator is then withdrawn, leaving the sheath in place. A ureteroscope can then be advanced through the sheath to access the ureter.

One problem with conventional ureteral access sheaths is that it is difficult to pass the sheath and dilator through the urinary tract given that the outer diameter of the sheath is significantly larger than the inner diameter of the passageways of the urinary tract. This is particularly true with respect to the intramural ureter, which typically comprises the narrowest section of the urinary tract. In addition, it is possible to damage the urinary tract during the insertion process by, for example, perforating a vessel of the tract, such as the urethra or the ureter.

Although a simple solution to the above-described problems would be to reduce the diameter of the access sheath to a point at which the sheath can be easily inserted, such a measure would reduce the inner diameter of the sheath to a point at which a typical ureteroscope may not fit within the access sheath, thereby defeating the purpose of using the access sheath.

### BRIEF DESCRIPTION OF THE DRAWINGS

The disclosed access sheath can be better understood with reference to the following drawings. The components in the drawings are not necessarily to scale.

FIG. 1 is a perspective view of an embodiment of an access sheath and means for expanding the access sheath.

FIG. 2 is a cross-sectional view of a portion of a first embodiment of a tube of the access sheath shown in FIG. 1.

FIG. 3 is a cross-sectional view of a portion of a second embodiment of a tube of the access sheath shown in FIG. 1.

FIG. 4 is an end view of an embodiment of a tube of the access sheath shown in FIG. 1 prior to expansion.

FIG. 5 is an end view of the tube of FIG. 4, shown after expansion.

FIG. 6 is an end view of an alternative embodiment of an access sheath, shown after expansion.

FIG. 7 is an end view of the sheath of FIG. 6, shown prior to expansion.

### DETAILED DESCRIPTION

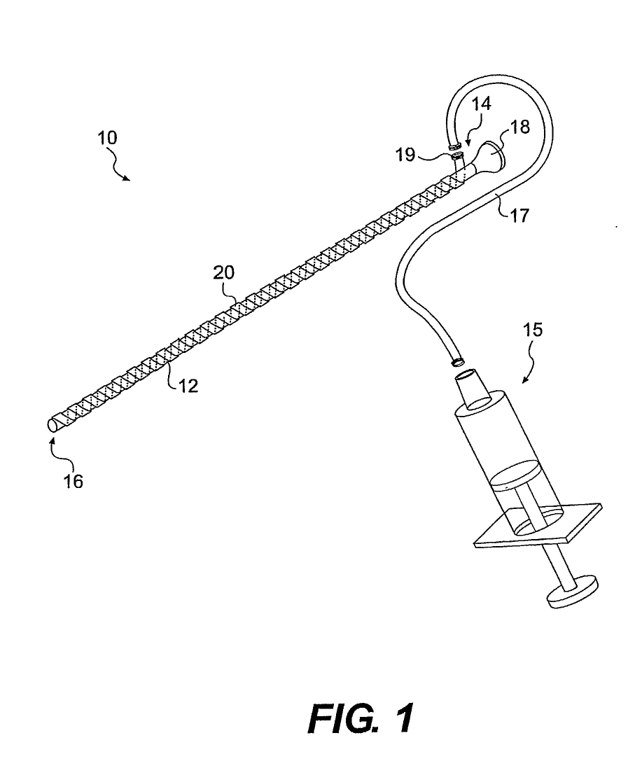

FIG. 1 illustrates an example embodiment of a ureteral access sheath 10 in an expanded state. As is described in greater detail below, the sheath 10 can be inserted into the urinary tract in an unexpanded state in which it has a small diameter, and can then be expanded while within the urinary tract to provide a passageway for a surgical instrument, such as a ureteroscope.

As is indicated in FIG. 1, the sheath 10 comprises an elongated lumen 12 having a proximal end 14 and a distal end 16. Positioned at the proximal end 14 of the lumen 12 is a lug 18 that forms an entrance to a working channel of the lumen.

The lumen 12 is made of a non-elastomeric material, such as polyethylene terephthalate (PET) or another suitable polymeric material, such as nylon. The walls of the lumen 12 are relatively thin. By way of example, the lumen walls are about 0.1 to about 2 millimeters (mm) thick. In its unexpanded state, the lumen 12 has an outer diameter (or French) that permits the lumen to be easily passed through the urinary tract of a patient. By way of example, the lumen 12 has an outer dimension of about 3 French (Fr.) in the unexpanded state. The lumen 12 has a length that, when the lumen is inserted in the urinary tract, is long enough to provide access to the upper ureter. By way of example, the lumen 12 is about 35 to about 55 centimeters (cm) long.

The lug 18 is formed of a rigid material, such as a plastic material or metal material, and, in some embodiments, can be used to couple the sheath 10 to other objects, such as a surgical instrument.

Formed around the lumen 12 is a balloon 20. In the embodiment shown in FIG. 1, the balloon 20 is formed as a continuous, elongated coil that is helically wrapped around the lumen 12. The balloon 20 defines a continuous interior chamber into which fluid can be introduced to inflate the balloon. Suitable fluids include gases, such as air, and liquids, such as saline-based solutions (e.g., that contain contrast media). Irrespective of the fluid that is used to inflate the balloon 20, such inflation causes the balloon to expand such that the inner and outer diameters defined by the coils of the balloon increase in size. Because the balloon 20 is attached (e.g., adhered or welded) to the outer surface of the lumen 12, expansion of the balloon results in similar expansion of the lumen, thereby enlarging the inner diameter of the lumen to provide space for an instrument, such as a ureteroscope, to pass.

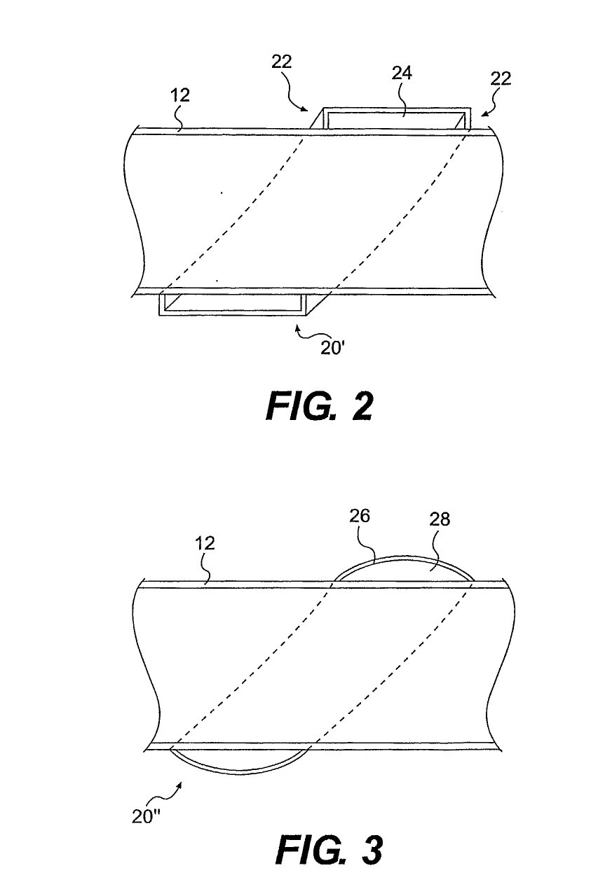

FIGS. 2 and 3 illustrate example embodiments for the balloon 20 shown in FIG. 1. Beginning with FIG. 2, illustrated is a balloon 20′ that has a rectangular cross-section that is defined by opposed square corners 22. The balloon 20′ defines an interior chamber 24 that, as is indicated by hidden lines, spirals around the lumen 12.

Turning to FIG. 3, illustrated is a balloon 20″ that has a rounded outer surface that is defined by a curved outer wall 26. Like balloon 20′, the balloon 20″ defines an interior chamber 28 that spirals around the lumen 12.

Irrespective of the particular configuration that is used for the balloon 20, the balloon is constructed of a non-elastomeric material that, although flexible, will not stretch as the balloon is pressurized. By way of example, the balloon 20 is constructed of PET or another suitable polymeric material. When such a material is used, the walls of the balloon 20 can be very thin. By way of example, the walls of the balloon 20 are about 0.003 inches (in) thick.

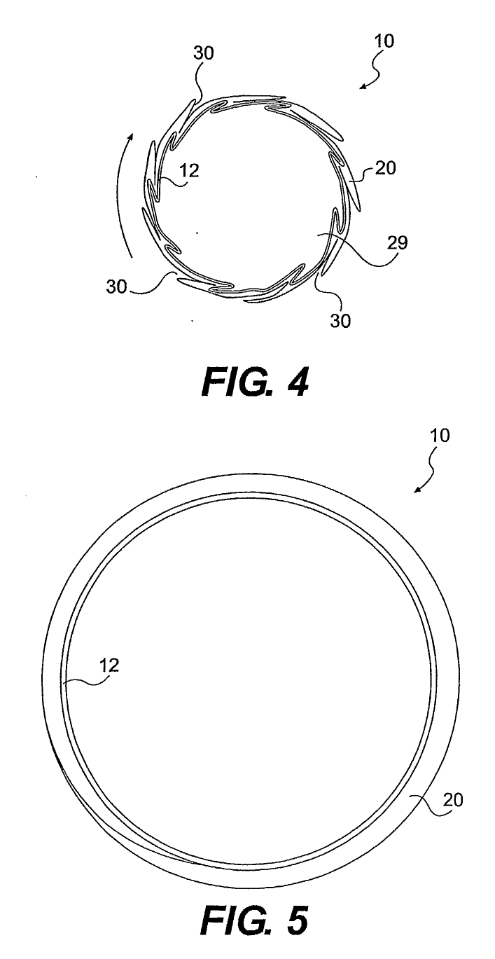

As is noted above, the sheath 10 is inserted into the urinary tract when the sheath is in an uninflated state in which the sheath has a relatively small outer diameter. In order to minimize that diameter, both the lumen 12 and the balloon 20 can be configured in an initial collapsed orientation. FIG. 4 illustrates an example collapsed orientation for the lumen 12 and the balloon 20. As is indicated in FIG. 4, the lumen is radially folded such than an inner passage 29 remains that is sized and configured to be passed over a guidewire. By way of example, the passage 29 has a diameter of about 0.016 in. to about 0.045 in. The balloon 20 is wrapped around the lumen 12 by forming a plurality of longitudinal creases 30 in the balloon. With such creases, the balloon 20 can be tightly wrapped around the lumen 12 in a selected direction (clockwise in FIG. 4). Once wrapped in this manner, the sheath 10 has a small effective outer dimension, for example about 3 Fr. to about 6 Fr.

A guidewire (not shown) may be used to facilitate insertion of the sheath 10. In such a case, the guidewire is first introduced into the urinary tract and is extended into the ureter. Once the guidewire has been so positioned, the sheath 10 can be passed over the guidewire to position the catheter within urinary tract and, typically, within the ureter.

Once the sheath 10 has been placed in the desired position along the urinary tract, a pathway has been established that protects the walls of the patient's urinary system. Given that the inner diameter of the lumen 12 is so small, however, the lumen must be expanded in order to provide a passageway that is large enough for an instrument, such as a ureteroscope, to pass. By way of example, such a ureteroscope may have an outer dimension of about 10 Fr.

As is described above, the lumen 12 is expanded by inflating the balloon 20. Referring back to FIG. 1, such inflation can be achieved using a syringe 15 or other appropriate inflation mechanism. In cases in which a syringe 15 is used, the inflation fluid can be delivered from the syringe through a supply tube 17 that connects to an inlet 19 of the balloon 20 that provides access to the balloon's interior chamber. Optionally, the syringe 15 can comprise a threaded plunger that enables precise dilation pressures to be reached and maintained.

When the balloon 20 is fully inflated and the sheath 10 is placed in the fully expanded state, the lumen 12 has an inner dimension of about 10 Fr. to about 14 Fr. FIG. 5 illustrates the fully expanded state. Due to the helical or spiral shape of the balloon 20, the balloon has good radial integrity that prevents the lumen 12 from collapsing or kinking, yet is still flexible such that the sheath 10 can follow the contours of the patient's urinary tract.

At this point, a passageway to the patient ureter has been formed that is large enough to pass instruments, such as a ureteroscope, through to the ureter. Given that the sheath 10 is inserted prior to expansion, insertion is easier to achieve and is less likely to cause damage to the patient's urinary system.

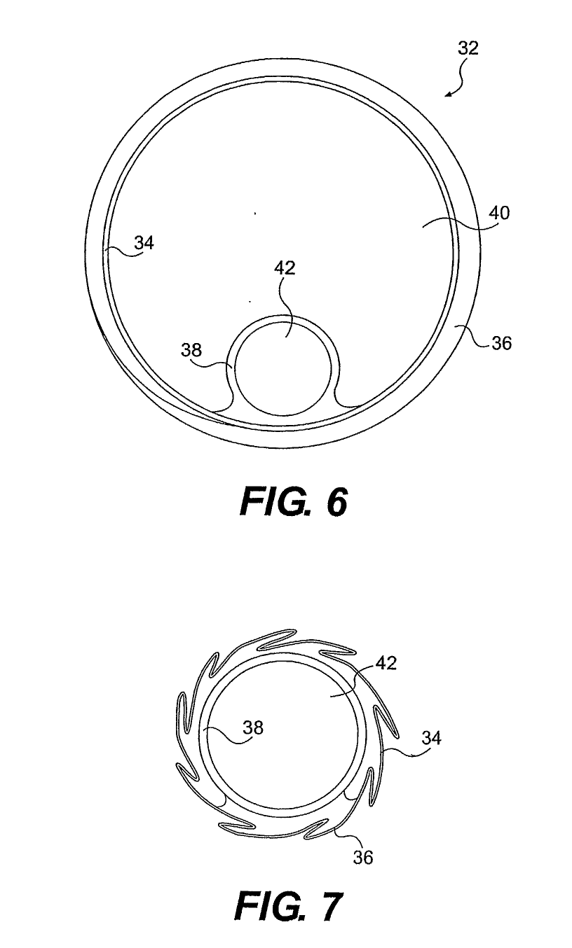

FIGS. 6 and 7 illustrate an alternative embodiment of an access sheath 32. As is indicated in FIG. 6, the access sheath 32, like access sheath 10, includes a lumen 34 and a balloon 36 that is helically wrapped around the lumen. In addition, however, the sheath 32 comprises an auxiliary lumen 38. With such an arrangement, the sheath 32 includes a main channel 40 that can be used as a passageway for a tool, such as a ureteroscope, and an auxiliary channel 42 can be used to receive a guidewire, to facilitate irrigation, or for another purpose. Notably, multiple auxiliary channels could be provided, if desired.

With reference to FIG. 7, the lumen 34 and balloon 36 can be wrapped around the auxiliary lumen 38 when the sheath 32 is in the unexpanded state. In such a case, the auxiliary channel 42 is sized to configured to receive the guidewire during insertion of the sheath 32.

While particular embodiments of the disclosed ureteral access sheath have been disclosed in detail in the foregoing description and drawings for purposes of example, variations and modifications thereof can be made. All such variations and modifications are considered to fall within the scope of the present disclosure.

1. An access sheath comprising:
a lumen defining an inner passage and an outer surface; and
a balloon that is provided on the lumen outer surface;
wherein when the balloon is inflated the balloon expands the lumen to increase the size of the inner passage.

2. The sheath of claim 1, wherein the lumen is sized and configured to extend from a position outside of a patient, through the uretha, and to an upper ureter of the patient.

3. The sheath of claim 1, wherein the balloon is formed as a continuous coil that is helically wrapped around the lumen.

4. The sheath of claim 3, wherein the balloon has a rectangular cross-section.

5. The sheath of claim 3, wherein the balloon has a round outer surface defined by a curved outer wall.

6. The sheath of claim 1, wherein the lumen and the balloon are provided in an initial collapsed orientation in which the lumen and balloon are folded such that the inner passage is sized and configured for a guidewire during insertion of the sheath within a patient vessel.

7. The sheath of claim 6, wherein the inner passage has a diameter in the initial collapsed orientation of about 0.016 to about 0.045 inches.

8. The sheath of claim 6, wherein the sheath has an outer dimension in the initial collapsed orientation of about 3 French to about 6 French.

9. The sheath of claim 1, wherein the inner passage has an inner dimension of about 10 French to about 14 French when the balloon is fully inflated.

10. The sheath of claim 1, wherein the lumen comprises a main channel and an auxiliary lumen provided within the main channel.

11. The sheath of claim 10, wherein the lumen and the balloon are provided in an initial collapsed orientation in which the lumen and balloon are folded and wrapped around the auxiliary lumen such that the inner passage of the sheath is a passage that extends through the auxiliary lumen.

12. The sheath of claim 11, wherein the auxiliary lumen is sized and configured for a guidewire during insertion of the sheath within a patient vessel.

13. The sheath of claim 1, further comprising a lug provided at a proximal end of the lumen that forms an entrance to the lumen.

14. A ureteral access sheath comprising:
an elongated lumen defining an inner passage and an outer surface; and
a balloon provided on the lumen outer surface and formed as a continuous coil that is helically wrapped around the lumen such that when the balloon is inflated the balloon expands the lumen to increase the size of the inner passage;
wherein the sheath comprises an initial collapsed orientation in which the lumen and balloon are folded such that the inner passage is sized and configured for passage of a guidewire and the sheath has an outer dimension that enables easy insertion of the sheath through a patient urethra, and an expanded orientation in which the inner passage is sized and configured for passage of a ureteroscope and the sheath has an outer dimension that dilates the urethra.

15. The sheath of claim 14, wherein the inner passage has a diameter in the initial collapsed orientation of about 0.016 to about 0.045 inches in the initial collapsed orientation.

16. The sheath of claim 14, wherein the sheath has an outer dimension in the initial collapsed orientation of about 3 French to about 6 French.

17. The sheath of claim 14, wherein the inner passage has an inner dimension in the expanded orientation of about 10 French to about 14 French.

18. A ureteral access sheath comprising:
an elongated lumen defining an outer surface, a main channel, and an auxiliary lumen provided in the main channel, the auxiliary lumen defining an inner passage sized and configured for passage of a guidewire; and
a balloon provided on the lumen outer surface and formed as a continuous coil that is helically wrapped around the lumen, the balloon expanding the main channel of the lumen when inflated;
wherein the sheath comprises an initial collapsed orientation in which the lumen and balloon are folded and wrapped around the auxiliary lumen such that the sheath has an outer dimension that enables easy insertion of the sheath through a patient urethra, and an expanded orientation in which the main channel is sized and configured for passage of a ureteroscope and the sheath has an outer dimension that dilates the urethra.

19. The sheath of claim 18, wherein the inner passage has a diameter in the initial collapsed orientation of about 0.016 to about 0.045 inches.

20. The sheath of claim 18, wherein the sheath has an outer dimension in the initial collapsed orientation of about 3 French to about 6 French.

21. The sheath of claim 18, wherein the main channel has an inner dimension in the expanded orientation of about 10 French to about 14 French.

22. A surgical method comprising:
passing an access sheath through a patient vessel when the sheath is in an initial collapsed orientation in which the sheath has a relatively small outer dimension and an inner passage of the sheath is relatively narrow; and
expanding the access sheath when placed in a desired position within the patient vessel so that the sheath has a relatively large outer dimension that dilates the patient vessel and the inner passage is expanded to provide a passageway of sufficient size to receive a surgical device that could not be received by the inner passage when the sheath was in the initial collapsed orientation; and
wherein the expandable nature of the access sheath enables easy insertion of sheath while also providing a passageway large enough for the surgical device to pass.

23. The method of claim 22, wherein passing the sheath through a patient vessel comprises passing the sheath over a guidewire already provided in the patient vessel, the guidewire being disposed within the inner passage.

24. The method of claim 23, further comprising withdrawing the guidewire from the inner passage once the sheath is placed in the desired position within the patient vessel.

25. The method of claim 22, wherein expanding the access sheath comprises inflating a balloon provided on an outer surface of a lumen of the access sheath, the balloon being formed as a continuous coil that is helically wrapped around the lumen.

26. The method of claim 22, wherein the inner passage has a diameter in the initial collapsed orientation of about 0.016 to about 0.045 inches.

27. The method of claim 22, wherein has an outer dimension the sheath in the initial collapsed orientation of about 3 French to about 6 French.

28. The method of claim 22, wherein a lumen of the access sheath has an inner dimension of about 10 French to about 14 French when the sheath is expanded.

29. The method of claim 22, wherein the surgical device is a ureteroscope.
